(19) United States
(12) Patent Application Publication
(10) Pub. No.: US 2018/0110822 A1
(43) Pub. Date: Apr. 26, 2018

(54) STABLE LIQUID PHARMACEUTICAL COMPOSITIONS OF BORTEZOMIB

(71) Applicant: LEIUTIS PHARMACEUTICALS PVT. LTD., Hyderabad (IN)

(72) Inventors: Kocherlakota CHANDRASHEKHAR, Secunderabad (IN); Banda NAGARAJU, Hyderabad (IN)

(73) Assignee: LEIUTIS PHARMACEUTICALS PVT. LTD., Hyderabad (IN)

(21) Appl. No.: 15/565,617

(22) PCT Filed: Apr. 12, 2016
(86) PCT No.: PCT/IB2016/052057

§ 371 Date: Oct. 10, 2017

(30) Foreign Application Priority Data
Apr. 13, 2015 (IN) 1908/CHE/2015
Jan. 7, 2016 (IN) 201641000573

(51) Int. Cl. A61K 38/05; A61K 47/10; A61K 47/26; A61K 47/22; A61K 47/20; A61K 47/18; A61K 9/00

(52) U.S. Cl. CPC A61K 38/05; A61K 47/10; A61K 47/26; A61K 9/0019; A61K 47/20; A61K 47/183; A61K 47/22

(57) ABSTRACT

The present invention relates to stable liquid pharmaceutical compositions of Bortezomib or a pharmaceutically acceptable salt thereof. Further the present invention relates to liquid parenteral formulations of Bortezomib comprising of Bortezomib, one or more solvents and other pharmaceutically acceptable adjuvants thereof.

### BACKGROUND OF THE INVENTION

Bortezomib is a modified di-peptidyl boronic acid. The chemical name for bortezomib, the monomeric boronic acid, is [(1R)-3-methyl-1-[[(2S)-1-oxo-3-phenyl-2-[(pyrazinyl carbonyl)amino]propyl]amino]butyl] boronic acid. The solubility of Bortezomib, as the monomeric boronic acid, in water is 3.3 to 3.8 mg/mL over a pH range of 2 to 6.5. The chemical structure of Bortezomib is shown below:

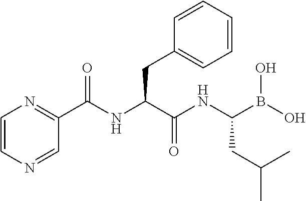 [SMILES: CC(C)C[C@H](NC(=O)[C@H](Cc1ccccc1)NC(=O)c1cnccn1)B(O)O]

Bortezomib is a reversible inhibitor of the chymotrypsin-like activity of the 26S proteasome in mammalian cells. The 26S proteasome is a large protein complex that degrades ubiquitinated proteins. The ubiquitin-proteasome pathway plays an essential role in maintaining homeostasis within cells by regulating the intracellular concentration of specific proteins. Inhibition of the 26S proteasome prevents this targeted proteolysis, which can affect multiple signaling cascades within the cell. This disruption of normal homeostatic mechanisms can lead to cell death.

Bortezomib is provided commercially as a mannitol boronic ester, which in reconstituted form consists of the mannitol ester in equilibrium with its hydrolysis product, the monomeric boronic acid. The drug substance exists in its cyclic anhydride form as a trimeric boroxine in solid state. Commercially, Bortezomib is sold as mannitol ester under the brand name Velcade® which is supplied as a sterile lyophilized powder for intravenous infusion and available in single-dose vials containing 3.5 mg of Bortezomib. The inactive ingredient is 35 mg mannitol, USP per vial. Velcade® when reconstituted forms a solution consisting of mannitol ester in equilibrium with Bortezomib. Velcade® is reconstituted with 0.9% sodium chloride to a final concentration of 1 mg/ml of Bortezomib.

U.S. Pat. Nos. 6,713,446 and 6,958,319 to Gupta Shanker et al., describe pharmaceutical compositions of boronic acid compounds prepared by lyophilizing an aqueous mixture comprising a boronic acid compound and a sugar that readily releases the boronic acid compound upon dissolution in aqueous media.

U.S Pat. No. 6,617,317 to Adams Julian et al., discloses a method for reducing the rate of degradation of proteins in an animal comprising contacting cells of the animal with certain boronic ester and acid compounds. Also disclosed are novel boronic ester and acid compounds, their synthesis and uses.

PCT Publication WO2010/089768 to Namdeo Alok et al., discloses pharmaceutical composition comprising Bortezomib and tromethamine in lyophilized form which is stable at room temperature and upon reconstitution forms aqueous solutions that are stable for at least 12 hours.

U.S Pat. No. 8,263,578 to Soppimath Kumaresh et al., describes liquid formulations comprising of Bortezomib and non-aqueous solvent system in which propylene glycol is a main component.

The use of propylene glycol has limited use in pediatric population. Even though propylene glycol is considered to be harmless, in high concentrations, adverse effects such as lactic acidosis and hyperosmolarity, hemolysis, renal toxicity including tubular dysfunction and acute tubular necrosis are reported. When propylene glycol is present in high doses in intravenous formulations, it increases the formulation osmolality. (EMA/CHMP/704195/2013) Hence its use in patients of diminished renal function should be monitored by determining plasmatic osmolality daily.

The reconstitution of Bortezomib lyophilized formulations is clinically inconvenient with implications of chemical instability. Further lyophilization makes the process more expensive and time consuming.

On the other hand, Bortezomib solutions formulated with propylene glycol may be stable but the use of propylene glycol presents acceptability limitations. Hence there is a need to develop alternate formulations of Bortezomib. The present invention addresses this need.

### SUMMARY OF THE INVENTION

The present invention relates to ready to use glycol free liquid formulations of Bortezomib intended for parenteral administration.

One aspect of the invention is to provide stable ready to use liquid pharmaceutical formulation comprising Bortezomib dissolved in suitable solvent or mixture of solvents, wherein the formulation is free of glycols.

Another aspect of the present invention is to provide stable ready to use liquid formulation comprising Bortezomib, chelating agents, suitable solvent or mixture of solvents and other pharmaceutically acceptable excipients thereof.

Yet another aspect of the present invention is to provide stable ready to use liquid formulation comprising of Bortezomib, chelating agents, stabilizers, suitable solvent or mixture of solvents and other pharmaceutically acceptable excipients thereof.

### DETAILED DESCRIPTION OF THE INVENTION

The term “Bortezomib” includes its pharmaceutically acceptable salts, solvates and hydrates thereof.

As used herein, “glycol” means class of organic chemicals characterized by having two hydroxyl (—OH) groups attached to separate carbon atoms; which may include dihydric alcohols such as ethylene glycol, propylene glycol, butylene glycol and polyalkylene glycols such as polyethylene glycol, polypropylene glycol, polybutylene glycol and the like.

The phrase “glycol free” as used herein generally means that the described composition comprises glycols less than about 10% by weight, based on the total weight of the formulation.

As used herein, “ready to use” Bortezomib formulations refers to formulations that contain Bortezomib in dissolved or solubilized form and are to be intended to be used as such or upon further dilution in intravenous diluents.

The pharmaceutical formulations of the present invention are intended for parenteral administration.

The pharmaceutical formulation of the present invention is a stable ready to use liquid formulation that is free of glycols. The inventors have surprisingly found that it is possible to develop stable liquid formulations of Bortezomib overcoming the disadvantages associated with prior art.

One embodiment of the invention relates to stable ready to use liquid formulation of Bortezomib comprising:
i. Bortezomib
ii. Chelating agents
iii. One or more solvents and optionally other pharmaceutically acceptable adjuvants thereof.

Another embodiment of the invention relates to stable ready to use liquid formulation of Bortezomib comprising:
i. Bortezomib
ii. Chelating agents
iii. Stabilizers
iv. One or more solvents and optionally other pharmaceutically acceptable adjuvants thereof.

Yet, another embodiment of the invention relates to stable ready to use liquid formulation of Bortezomib comprising:
i. Bortezomib
ii. Chelating agents selected from the group comprising DOTA, DTPA and EDTA
iii. One or more solvents selected from the group comprising ethanol, glycerine and water
iv. Anti-oxidants selected from the group comprising monothioglycerol, sodium metabisulfite and L-cysteine
v. Optionally stabilizers such as sugars and aminoacids.

A preferred embodiment of the invention comprises:
i. Bortezomib
ii. DOTA
iii. One or more solvents such as ethanol, glycerine and water
iv. Monothioglycerol and
v. Optionally stabilizers such as mannitol, sorbitol, arginine, glycine and lysine.

Suitable solvents can be selected from aqueous and non-aqueous solvents such as, but are not limited to, glycerine, ethanol, n-propanol, n-butanol, isopropanol, ethyl acetate, dimethyl carbonate, acetonitrile, dichloromethane, methyl ethyl ketone, methyl isobutyl ketone, cyclohexane, dimethylacetamide (DMA), dimethyl sulfoxide (DMSO), N-Methyl-2-pyrrolidone (NMP), 1,3-dimethyl-2-imidazolidinone (DMI), acetone, tetrahydrofuran (THF), dimethylformamide (DMF), propylene carbonate (PC), dimethyl isosorbide, water and mixtures thereof. Preferred solvents are ethanol, glycerine and water.

The formulation of the present invention comprises stabilizers such as sugars and amino acids. Suitable stabilizers include glucose, trehalose, sucrose, mannitol, sorbitol, arginine, glycine, proline, methionine, lysine and the like.

Suitable chelating agents include, but not limited to DOTA (1,4,7,10-tetraazacyclododecane-1,4,7,10-tetraacetic acid), DTPA (diethylene triaminepentaacetic acid), EDTA (Ethylenediaminetetraacetic acid), ODDA (1,4,10,13-tetraoxa-7,16-diazacyclooctadecane-7), TTTA (1,7,13-triaza-4,10,16-trioxacyclooctadecane-N,N′,N″-triacetate), DOTRP (tetraethyleneglycol-1,5,9-triazacyclododecane-N,N′,N″,-tris(methylene phosphonic acid), EGTA (ethylene glycol-bis(β-aminoethyl ether)-tetraacetic acid) and the like.

The pharmaceutical compositions of the present invention also contain one or more anti-oxidants. Suitable anti-oxidants include, but are not limited to monothioglycerol, ascorbic acid, sodium bisulfite, sodium metabisulfite, L-cysteine, thioglycolic acid, citric acid, tartaric acid, phosphoric acid, gluconic acid, thiodipropionic acid and the like. Most preferred anti-oxidant is monothioglycerol.

The pharmaceutical compositions of the present invention optionally contain other pharmaceutically acceptable adjuvants such as buffering agents, pH adjusting agents, preservatives, tonicity modifiers and the like.

The invention further relates to a process of preparing ready to use liquid formulations of Bortezomib comprising
(i) Addition of Bortezomib to non-aqueous solvent
(ii) Addition of chelating agent and optionally anti-oxidants and stabilizers to the aqueous solvent
(iii) Addition of aqueous solvent to the non-aqueous solvent containing Bortezomib
(iv) Filtering and filling of the solution in suitable container or vials followed by stoppering and sealing of the vials.

Alternately, ready to use liquid formulations of Bortezomib can be prepared by the following process comprising
(i) Addition of anti-oxidant, chelating agent and optionally stabilizer to the aqueous solvent, followed by the addition of Bortezomib
(ii) Addition of stabilizer to the aqueous solvent followed by heating
(iii) Addition of solution of step (ii) to step (i)
(iv) Filtering and filling of the solution in suitable container or vials followed by stoppering and sealing of the vials.

Bortezomib formulation prepared according to the invention was tested for stability at various conditions such as 2-8° C., 25° C./60%RH, 30° C./65%RH and 40° C./75%RH for 2 months. The stability data of the invention formulation is summarized in Table 1.

TABLE 1
Stability Profile of the formulation prepared according to example 1
(3.5 mg/ml).

| | | Time Interval | | | | | | | |
|---|---|---|---|---|---|---|---|---|---|
| | | 1 M | 2 M | 1 M | 2 M | 1 M | 2 M | 1 M | 2 M |
| | Condition | | | | | | | | |
| | Initial | 2-8° C. | | 25° C./60% RH | | 30° C./65% RH | | 40° C./75% RH | |
| Assay | 100.1 | 100.8 | 100.8 | 100.2 | 98.4 | 100.3 | 96.4 | 96.1 | 95.2 |
| pH | 4.19 | 4.35 | 4.32 | 4.34 | 4.38 | 4.34 | 4.40 | 4.28 | 4.3 |
| Impurities | Related substances by HPLC (% w/w) | | | | | | | | |
| Total Impurities | 0.29 | 0.33 | 0.35 | 0.70 | 1.07 | 1.05 | 1.78 | 2.94 | 5.11 |

The following examples further describes certain specific aspects and embodiments of the present invention and demonstrates the practice and advantages thereof. It is to be understood that the examples are given by way of illustration only and are not intended to limit the scope of the invention in any manner.

### EXAMPLE 1

| S. No | Ingredients | | | | | | | | |
|---|---|---|---|---|---|---|---|---|---|
| Concentration | | Quantity per vial | | | | | | | |
| (mg/mL) | | 1 | | 2.5 | | 1.5 | | 3.5 | |
| 1. | Bortezomib | 3 | mg | 3 | mg | 3.0 | mg | 3.5 | mg |
| 2. | Ethanol | 0.045 | mL | 0.045 | mL | 0.045 | mL | 0.045 | mL |
| | (99%) | | | | | | | | |
| 3. | Mannitol | 30 | mg | 30 | mg | 30 | mg | 35 | mg |
| 4. | DOTA | 0.98 | mg | 0.98 | mg | 0.98 | mg | 0.98 | mg |
| 5. | Mono- | 0.17 | mg | 0.17 | mg | 0.17 | mg | 0.17 | mg |
| | thioglycerol | | | | | | | | |
| 6. | L-Arginine | 0.28 | mg | 0.29 | mg | 0.28 | mg | 0.28 | mg |
| 7. | Water for | Q.s 3 | mL | Q.s 1.2 | mL | Q.s 2.0 | mL | Q.s 1 | mL |
| | Injection | | | | | | | | |

### Manufacturing Process

Ethanol was taken into compounding vessel and monothioglycerol and Bortezomib were added and stirred. DOTA, mannitol and L-arginine were added to the compounding vessel containing water for injection and stirred to form a uniform solution. Water for injection solution was transferred to ethanol solution and stirred. The solution was filtered and filled into suitable containers. The vials were stored at 2°-8° C.

### EXAMPLE 2

| S. No | Ingredients | Quantity per vial | |
|---|---|---|---|
| 1. | Bortezomib | 3 | mg |
| 2. | Mannitol | 30 | mg |
| 3. | DOTA | 0.98 | mg |
| 4. | Monothioglycerol | 0.17 | mg |
| 5. | L-Arginine | 0.28 | mg |
| 6. | Water for Injection | Q.s 1.2 | mL |

### Manufacturing Process

Monothioglycerol, mannitol and DOTA were added to water followed by the addition of Bortezomib (Mixture I). Arginine was dissolved in water and heated. (Mixture II). The obtained Arginine solution was added to mixture I and stirred till a clear solution was obtained. The solution was filtered and filled into suitable containers.

### EXAMPLE 3

| | | |
|---|---|---|
| S. No | Ingredients | Quantity per vial |

| | | | |
|---|---|---|---|
| 1. | Bortezomib | 3.5 mg | 3.5 mg |
| 2. | Glycerine | 126 mg | 264.6 mg |
| 3. | Ethanol | 39.45 mg | 78.9 mg |
| 4. | DOTA | 0.035 mg | — |
| 5. | Monothioglycerol | 1 mg | 1 mg |
| 6. | Water for injection | 350 mg | 190 mg |

### Manufacturing Process

Required quantity of ethanol was taken into compounding vessel and Bortezomib was added and stirred, followed by the addition of glycerine. Required quantity of water for injection was taken in another vessel and DOTA and Monothioglycerol were added and stirred to form a uniform solution. Both the solutions were mixed and stirred to get a uniform solution. The obtained solution was filtered and filled into the vials.

### EXAMPLE 4

| S. No | Ingredients | Quantity per vial | | |
|---|---|---|---|---|
| 1. | Bortezomib | 3.50 mg | 3.50 mg | 3.50 mg |
| 2. | Glycerine | 260 mg | 260 mg | 260 mg |
| 3. | Ethanol | 200 mg | 170 mg | 160 mg |
| 4. | DOTA | 0.98 mg | 0.98 mg | 0.98 mg |
| 5. | Monothioglycerol | 0.17 mg | 0.17 mg | 0.17 mg |
| 6. | Water for Injection | 40 mg | 75 mg | 90 mg |

### Manufacturing Process

Required quantity of ethanol was taken into compounding vessel and Bortezomib was added and stirred, followed by the addition of glycerine. Required quantity of water for injection was taken in another vessel and DOTA and Monothioglycerol were added and stirred to form a uniform solution. Both the solutions were mixed and stirred to get a uniform solution. The obtained solution was filtered and filled into the vials.

We claim:

1: A stable, ready to use liquid formulation comprising of Bortezomib and pharmaceutically acceptable adjuvants, wherein the formulation is free of glycols.

2: A stable, ready to use liquid formulation comprising
(i) Bortezomib
(ii) Chelating agents
(iii) One or more solvents and optionally other pharmaceutically acceptable adjuvants thereof.

3: The pharmaceutical formulation according to claim 2, comprising
(i) Bortezomib
(ii) Chelating agents selected from the group comprising DOTA, DTPA and EDTA
(iii) One or more solvents such as ethanol, glycerine and water and
(iv) Optionally stabilizers such as sugars and amino acids.

4: The ready to use liquid pharmaceutical formulation of claim 3, which further comprises an antioxidant.

5: The ready to use liquid pharmaceutical formulation of claim 4, where the antioxidant is selected from the group comprising butylated hydroxyanisole (BHA), butylated hydroxyl toluene (BHT), citric acid, monothioglycerol, ascorbic acid and cysteine.

6: A stable, ready to use liquid formulation of Bortezomib comprising
(i) Bortezomib
(ii) Chelating agents such as DOTA, DTPA and EDTA
(iii) One or more solvents such as ethanol, glycerine and water
(iv) Stabilizers such as mannitol, sorbitol, arginine, glycine and lysine
(v) Anti-oxidants such as monothioglycerol and optionally other pharmaceutically acceptable excipients thereof.
